(19) United States
(12) Patent Application Publication
(10) Pub. No.: US 2004/0219588 A1
(43) Pub. Date: Nov. 4, 2004

(54) Method for dispensing reagents onto biological samples and method for analyzing biological samples

(76) Inventor: Masaru Furuta, Kyoto-shi (JP)

Correspondence Address: DARBY & DARBY P.C., P. O. BOX 5257, NEW YORK, NY, 10150-5257, US

(21) Appl. No.: 10/834,203

(22) Filed: Apr. 29, 2004

(30) Foreign Application Priority Data
Apr. 30, 2003 (JP) 2003-125805

(51) Int. Cl. C12Q 1/68; C12M 1/34; B05D 3/00

(52) U.S. Cl. 435/6; 435/287.2; 427/2.11

(57) ABSTRACT

The present invention provides a method for locally dispensing a controlled nanoliter volume of a reagent onto a several hundred micron-size region at any desired location on a biological sample, as well as an analytical method for analyzing a biological sample that uses the aforementioned method. A method for dispensing a reagent onto a biological sample, wherein a reagent solution is delivered, by using inkjet technology, to a particular region on a biological sample containing cells or tissues, to make the particular region spotted with the reagent solution. A method for analyzing a biological sample containing cells or tissues, comprising the steps of: dispensing a reagent onto a biological sample for delivering a reagent solution, by using inkjet technology, to a particular region on the biological sample containing cells or tissues, to make the particular region spotted with the reagent solution; subsequently analyzing a target material present in the cells or the tissues by microscopy or mass spectrometry.

### BACKGROUND OF THE INVENITION

1. Field of the Invention

The present invention relates to various medical and biological fields, including cell biology, pathology, and biochemistry. Specifically, the present invention relates to a method for dispensing reagents onto biological samples to allow the reagents to react with the samples and a method for detecting the reaction products.

2. Disclosure of the Related Art

Microscopy is commonly used in the analysis of biological samples, such as cells and tissues. When it is desired to identify target materials (e.g., protein molecules) using microscopy, specific antibodies are intervened and used to label the target materials, and the resulting color, luminescence, or fluorescence is detected. In recent years, mass spectrometric approaches have been proposed in which laser is directly irradiated onto sample sections to obtain a mass spectrum as described in for example Japanese Patent Laid-Open Publication No. 2001-249125. Furthermore, for the purpose of determining the local distribution of target materials, various staining techniques using labels such as those described above have been used, as have imaging techniques based on MALDI mass spectrometry as described in for example U.S. Pat. No. 5,808,300, and techniques based on genetic recombination in which a fluorescent protein is co-expressed with a target protein.

On the other hand, in Japanese Patent Laid-Open Publication No. Hei 11-187900, it is described about a technique in which a liquid containing a probe is dispensed onto a solid phase using inkjet technology to manufacture a probe array.

### SUMMARY OF THE INVENTION

In each of the above-described techniques for the analysis of biological samples, it is difficult to minimize the amounts of reagents used to treat samples since it is either the whole sample or a limited region of a sample that needs to be treated. In cases where limited regions of a sample are to be treated, such regions are separated from other regions of the sample by for example a water-repellent rubber. Also, when it is desired to analyze a particular small area of a sample, the signals generated from adjacent areas serve as a source of background noise that interfere with the analysis. Thus, none of the conventional techniques are suitable for the purpose of applying different reagents or different analytical techniques to different regions of a sample.

It is thus an objective of the present invention to provide a method for locally dispensing a controlled nanoliter volume of a reagent onto a several hundred micron-size region at any desired location on a biological sample, as well as an analytical method for analyzing a biological sample that uses the aforementioned method.

In the course of studies, the present inventor has found that the above-described objective can be attained by the use of inkjet technology and have thus completed the present invention.

The present invention comprises the following inventions:

(1) A method for dispensing a reagent onto a biological sample, wherein a reagent solution is delivered, by using inkjet technology, to a particular region on a biological sample containing cells or tissues, to make the particular region spotted with the reagent solution.

(2) The method according to above (1), wherein microstructures of the cells or the tissues are preserved in the same forms as are found in vivo.

(3) The method according to above (1) or (2), wherein the cells and the tissues of the biological sample are embedded in an embedding material.

(4) The method according to any of above (1) to (3), wherein the embedding material is selected from the group consisting of water, paraffin, celloidin, carbowax, gelatin, albumin, agarose, epoxy resin, polyester resin, and glycol methacrylate.

(5) The method according to any of above (1) to (4), wherein the reagent is a compound that chemically reacts with a target material present in the cells or the tissues and/or a compound that specifically binds to the target material present in the cells or the tissues.

(6) The method according to above (5), wherein following the delivery of the reagent solution for spotting to the biological sample, the reagent chemically or biologically reacts with the target material.

(7) The method according to above (5) or (6), wherein the compound that specifically binds to the target material present in the cells or the tissues comprises at least one biological compound selected from the group consisting of proteins, peptides, nucleic acids, sugars, and lipids, and/or an analogue of the biological compound that has the same or similar binding ability to the biological compound.

In the present invention, the term “nucleic acid” encompasses DNA and RNA.

(8) The method according to above (5) or (6), wherein the compound that specifically binds to the target material present in the cells or the tissues comprises at least one biological compound selected from the group consisting of antibodies, antigens, and enzymes, and/or an analogue of the biological compound that has the same or similar binding ability to the biological compound.

(9) The method according to any of above (5) to (8), wherein the compound that specifically binds to the target material present in the cells or the tissues comprises at least one selected from the group consisting of labeled forms of the biological compound labeled with a labeling compound, complexes that the biological compound forms with the labeling compound, labeled forms of the analogue labeled with the labeling compound, and complexes that the analogue forms with the labeling compound.

(10) The method according to above (9), wherein the labeling compound is selected from the group consisting of fluorescent compounds, enzymes, heavy metal-containing compounds, and radioisotope-containing compounds.

(11) The method according to above (10), wherein the fluorescent compound is fluorescein isothiocyanate and/or tetramethylrhodamine isothiocyanate.

(12) The method according to above (10), wherein the enzyme is selected from the group consisting of horseradish peroxidase, alkaline phosphatase, acidic phosphatase, glucose oxidase, and tyrosinase.

(13) The method according to above (10), wherein the heavy metal-containing compound is ferritin and/or colloidal gold.

(14) The method according to any of above (1) to (13), wherein the biological sample is immobilized onto a surface of a substrate selected from the group consisting of glass substrate, resin substrate, and metal substrate, the biological sample is transferred to a membrane, or the biological sample is transferred to the membrane and then the membrane transferred is fixed to the surface of the substrate.

(15) The method according to above (14), wherein the metal substrate is a sample plate for mass spectrometry.

(16) The method according to above (14) or (15), wherein the membrane is a synthetic organic polymer or a derivative thereof selected from the group consisting of polyvinylidene difluoride, nitrocellulose, polyamide, and polyethylene.

(17) A method for analyzing a biological sample containing cells or tissues, comprising the steps of:

dispensing a reagent onto a biological sample for delivering a reagent solution, by using inkjet technology, to a particular region on the biological sample containing cells or tissues, to make the particular region spotted with the reagent solution; and

subsequently analyzing a target material present in the cells or the tissues by microscopy or mass spectrometry.

The method for dispensing a reagent may be used the method of any of the above (1) to (16).

(18) The method according to above (17), wherein the microscopic analysis includes detecting a color, luminescence, or fluorescence of the target material, or the presence of a radioisotope in the target material.

(19) The method according to above (17), wherein the mass spectrometry is laser desorption ionization mass spectrometry.

According to the present invention, there is provided a method for locally dispensing a controlled nanoliter volume of a reagent onto a several hundred micron-size region at any desired location on a biological sample, as well as an analytical method for analyzing a biological sample that uses the aforementioned method.

### BRIEF DESCRIPTION OF THE DRAWINGS

FIG. 1 is a schematic diagram illustrating one example of the manner in which a reagent is dispensed in accordance with the present invention.

FIG. 2 is a schematic diagram illustrating one example of detection of a target material in accordance with the present invention.

FIG. 3 is a photograph of the frozen section of mouse brain on which the reagent is dispensed in accordance with the present invention, and a illustration of the dispensed positions.

FIG. 4 is mass resulting mass spectra (a) to (i) of respective close positioned microscale nine regions on the sample, obtained by the analysis in accordance with the present invention.

### DETAILED DESCRIPTION OF THE INVENTION

According to the present invention, a reagent solution is delivered onto a particular region of a biological sample using inkjet technology. Once delivered, the reagent is distributed to specific structures and their constituent molecules in the cells or the tissues of the biological sample. Thus, the term “dispense” as used herein means that the reagent is delivered to the sample in the form of droplets to allow the distribution of the reagent.

According to the present invention, target materials are first made into detectable forms by using the inkjet technology-based dispensing method and are then analyzed. The analysis is conducted using microscopy, mass spectrometry, or other analytical techniques.

The biological sample in the present invention comprises cells or tissues of an organism that are not subjected to any fixation treatment. Any cell or tissue in which microstructures are preserved in the same forms as are found in vivo can be used. Cellular or tissue microstructures may be preserved by embedding cells or tissues in a proper embedding material. Such an embedding material may be any proper material selected depending on the type of the observation technique used and the characteristics of the biological sample. Examples include water, paraffin, celloidin, carbowax, gelatin, albumin, agarose, epoxy resin, polyester resin, and various water-soluble resins such as glycol methacrylate. One skilled in the art may properly select the embedding material. Preferred biological samples include unfixed samples, such as frozen sections, and fixed samples, such as paraffin-embedded sections.

Since the method of the present invention can analyze biological samples in which microstructures are preserved in the same forms as are found in vivo, the localization pattern of a target material revealed by the method of the present invention can be considered to be the same as how the target material is localized in vivo. Thus, in vivo localization of the target material can be determined through morphological observation of the samples.

The biological samples may be immobilized onto the surface of a substrate, such as a glass substrate, a resin substrate or a metal substrate, or the samples may be electrically transferred to a membrane. Examples of the membrane include those made of synthetic organic polymers and derivatives thereof such as polyvinylidene difluoride (PVDF), nitrocellulose, polyamide, and polyethylene. Examples of the polyamide include nylon. When the analysis of the present invention is conducted by using mass spectrometry, a metal substrate, such as a sample plate for mass spectrometry, is preferably used as the substrate; and a biological sample may be immobilized onto the sample plate, or the sample may be transferred to a membrane and the membrane is fixed to the sample plate by for example sticking. To fix the membrane to the sample plate, an electroconductive double-sided adhesive tape may preferably be used.

When necessary, the sample may be subjected to a proper pretreatment process, such as staining, washing or desalting.

A reagent solution is then dispensed onto a particular region of the biological sample prepared in the manner described above. According to the present invention, the solution is derivered onto the particular region in the form of fine scale droplets by using inkjet technology as dispensing technique, to make the particular region spotted with the solution. A preferred inkjet mechanism for use in the present invention is the piezo jet technology described in U.S. Pat. No. 4,877,745 that employs a piezoelectric device, while other mechanisms may also be used. A dispensing apparatus for use is described in Publication No. WO98/47006 while other apparatuses may also be used.

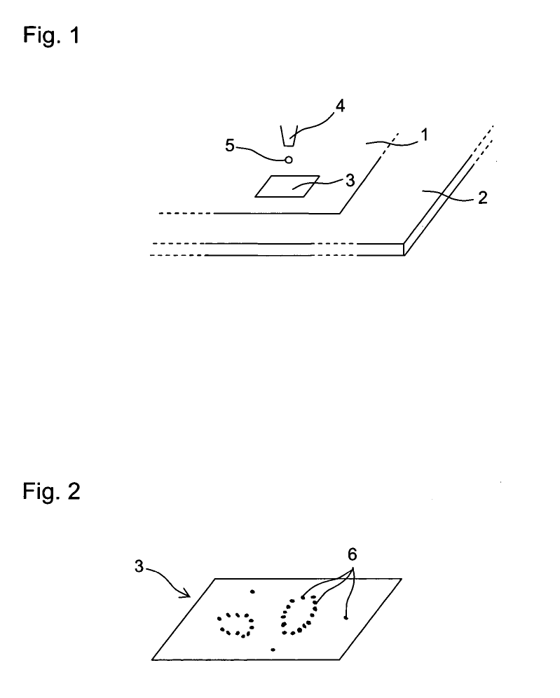

FIG. 1 schematically shows one embodiment of the present invention and illustrates how a reagent is dispensed in accordance with the present invention. A biological sample 1, or a membrane 1 to which a biological sample has been transferred, is fixed to a sample substrate 2 made of glass, a resin, or a metal or the like. A part of the biological sample 1 or the membrane 1 with the biological sample transferred thereto is designated as a particular region 3 onto which a reagent solution is to be dispensed. An inkjet-discharging element 4 is positioned above the region 3 and discharges a reagent droplet 5 onto the region 3.

In the dispensing method of the present invention, the use of inkjet technology allows the reagent to be dispensed in extremely small volumes. Specifically, the volume of the reagent discharged in each dispensing from each inkjet-discharging element may be adjusted to, for example, as small as 100 pl or to even smaller volumes depending on the inkjet mechanism employed. Also, as large an amount of the reagent as desired can be delivered by repeating the dispensing action multiple times. It is possible to dispense nanoliter volumes, typically approximately 100 nl, of the reagent onto a single region. In summary, the method of the present invention achieves minimum reagent consumption and, thus, a significant cost reduction even when expensive, hard-to-obtain reagents such as antibody solutions are used.

According to the present invention, a discharge volume of approximately 100 pl is typically dispensed onto an extremely small area of approximately 7800 μm². The area to which the reagent is delivered may be further reduced depending on the inkjet mechanism employed. Also, as large an area of the biological sample 1 as desired may be covered by moving the inkjet-discharging element 4 relative to the biological sample 1, since in this manner, the reagent solution can be delivered to any desired location on the biological sample 1. The area of a single region to which the reagent solution is delivered is for example about several square millimeters, and preferably up to about 10 mm².

According to the method of the present invention, different reagent solutions may be delivered to different regions on the same biological sample. Thus, simultaneous analysis of multiple species is possible.

In the present invention, minimum amounts of a reagent solution can be delivered to regions of minimum area on a single biological sample so that there is no need to construct physical partitions on the sample, as was the case with conventional techniques. For this reason, the time required in the analysis can be reduced and the damage to the sample caused by the partitions can be avoided.

In the present invention, the target material may be a particular tissue in a biological sample or a particular molecule in a tissue. Examples of the target material include antigens (including haptens), antibodies, and enzymes. Including these substances, examples of the target material further include generally proteins, peptides, nucleic acids, sugars, and lipids. The reagents for use in the present invention may be those that are capable of converting the target materials present in the biological sample into detectable forms so that the localization of the targets can be detected. The term “detectable” as used herein means that the target can be detected by visualization or mass spectrometry analysis.

In the present invention, compounds that can chemically react with the target material or compounds that can specifically bind to the target material may be used as the reagent to directly or indirectly detect the target material. The compound that can chemically react with the target material may be any compound that generates a color, luminescence, or fluorescent depending on the chemical properties of the target material and can be detected by ordinary techniques.

The compounds that can specifically bind to the target material may be either biological compounds or synthetic compounds. Examples of the biological compounds include proteins, peptides, nucleic acids, sugars, and lipids. In the present invention, specific binding ability of these biological compounds can be utilized. Specifically, specific binding between antigens and corresponding antibodies, between enzymes and corresponding substrates, and between nucleic acids and corresponding nucleic acids can be utilized. Thus, the compounds that can specifically bind to the target material may be provided as, for example, antibodies, antigens, or enzymes. The synthetic compounds for use in the present invention include analogues of biological compounds that have the same or similar binding abilities to the biological compounds. These analogues may be organic compounds or metal complexes. Aside from those that are structurally similar to the biological compounds, the analogues include those that have the same or similar binding abilities to the biological compounds. Examples of such analogues include modified forms of the biological compounds and enzyme models. Also, a label may be introduced into the compounds that can specifically bind to the target material. Specifically, the compounds that can specifically bind to the target material may be labeled compounds labeled with labeling compounds, or complexes formed with labeling compounds.

In the present invention, immunohistochemical approaches may be employed in order to make the target material in the biological sample detectable when the target material is an antigen (including haptens). Such antigens may be proteins, peptides, nucleic acids, sugars, and lipids. The immunohistochemical technique used for this purpose may be any commonly used technique, including fluorescent antibody technique, enzyme antibody technique, heavy metal antibody technique, and radiolabeled antibody technique. These techniques may be either labeled antibody techniques or non-labeled antibody techniques, or they may be either direct antibody techniques or indirect antibody techniques. Specifically, the reagents for use in the present invention may be primary antibodies or secondary antibodies, which are provided in the form of labeled or non-labeled proteins or peptides. When it is desired to visualize the antibodies by staining, various stains are also included in the reagents.

The labeling compounds include fluorescent compounds, enzymes, heavy metal-containing compounds, radioisotope-containing compounds, and other compounds. Examples of the fluorescent compounds include fluorescein isothiocyanate (FITC) and tetramethylrhodamine isothiocyanate (TRITC). Examples of the enzymes include horseradish peroxidase, alkaline phosphatase, acidic phosphatase, glucose oxidase, and tyrosinase. Examples of the heavy metal-containing compounds include ferritin, and colloidal gold. Examples of other compounds include enzyme models. Biotin may be used as the labeling compound. Labeled biotin/avidin complex and labeled streptavidin may also be used.

These reagents may be used either individually or as a mixture of two or more reagents.

The target materials in the biological sample may be made detectable by using techniques selected depending on the type of the antibodies or the labels used to label the antibodies. Specifically, the target materials are detected by the presence of colors, luminescence, fluorescence, and radioisotopes. Once made into a detectable form, the target material in the biological sample is analyzed by microscopy or mass spectrometry.

FIG. 2 shows one example of the detection of the target material and schematically illustrates an enlarged view of the region. 3 shown in FIG. 1. The reagent solution has been dispensed onto the region 3. In the region 3, the signals 6 of visualized target material are verified. The localization of the signals is observed using for example a microscope.

In performing microscopy, for example, enzymes may be visualized by dispensing a corresponding substrates and using calorimetric assay or luminescent assay techniques. Observation may be made with a light microscope. Fluorescent materials may be visualized by irradiating a proper excitation light to cause the materials to emit fluorescence. Observation may be made using a fluorescent microscope or a laser microscope Heavy metal-containing compounds may be visualized by the scattering of incident light, and observation may be made with an electron microscope. Radioisotopes can be visualized and observed by autoradiography.

In the present invention, enzyme histochemical approaches may be employed in order to make the biological material as the target material present in the biological sample detectable when the target material is an enzyme. The reagent in this case is a proper substrate for the target enzyme, and the target enzyme may be verified using any commonly used technique.

In the present invention, in situ hybridization may be employed in order to make the biological material as the target material present in the biological sample detectable when the target material is a nucleic acid (such as DNA and RNA). The reagent in this case is a labeled DNA probe that can hybridize the nucleic acid containing the targeted gene. The target nucleic acid may be verified using any commonly used technique. The labeling compound of the labeled DNA probe include, aside from those described with reference to the immunohistochemical approaches above, those recommended by the skilled in the art.

In performing mass spectrometry, laser desorption ionization mass spectrometry may be used. In this technique, the target material made into a detectable form is subjected to the following processes: First, the target material is digested, if necessary. This is done by adding a solution of a protease, such as trypsin, to the material and incubate the material under wet conditions. The optionally treated target material is then irradiated with laser to obtain a mass spectrum. Alternatively, a matrix solution may be dispensed onto the optionally treated target material and laser is subsequently irradiated onto the applied matrix to obtain a mass spectrum. According to the present invention, the reagent is dispensed onto a fine scaled area so that resulting mass spectrum is less susceptible to unwanted signals from the surrounding regions, or background noise. Also, by repeating the dispensing of the matrix solution and the subsequent laser irradiation to cover, rather than one small point on the sample, an area of predetermined size, a particular cell type or tissue can be imaged based on the mass distribution and the target material can be mapped on the biological sample.

As set forth, the method of the present invention, by which a reagent is dispensed onto a limited fine scaled area on a biological sample, can significantly reduce the reagent consumption as compared to conventional dispensing techniques. While it is expected that the biological samples to be analyzed include many valuable pathological tissue samples, the method of the present invention can make more effective use of the biological samples by allowing simultaneous analysis of multiple species on the same sample. Also, when applied in mass spectrometry, the method of the present invention achieves significant reduction in the amount of signals that are generated from the surrounding regions other than the regions dispensed and serve as a source of background noise.

### EXAMPLES

The present invention will be described in more detail referring to embodiments, but the present invention should not be limited to these embodiments. In the present example, the analysis of microscale region in a tissue section sample by laser desorption ionization-time of flight mass spectrometry (MALDI-TOF MS) is described.

For sample preparation, the frozen section of mouse brain was blotted on the PVDF membrane. This membrane was stained with Direct Blue 71, and attached on the MALDI target plate using the double-sided conductive tape. Microscale regions for dispensing were set on the tissue sample, 7 nL of polyvinyl-pyrrolidone solution was dispensed onto each position of the microscale regions and followed by dispensing 50 nL of 200 μg/mL trypsin solution by using the Chemical Inkjet Printer (CHIP-1000, SHIMADZU CORPORATION). After overnight incubation of this sample in the humidified condition at 30° C., 100 nL of the matrix solution (10 mg/mL CHCA: α-cyano-4-hydroxy cinnamic acid) was dispensed on the same position. Resulting dispensed areas were about 400 μm in diameter.

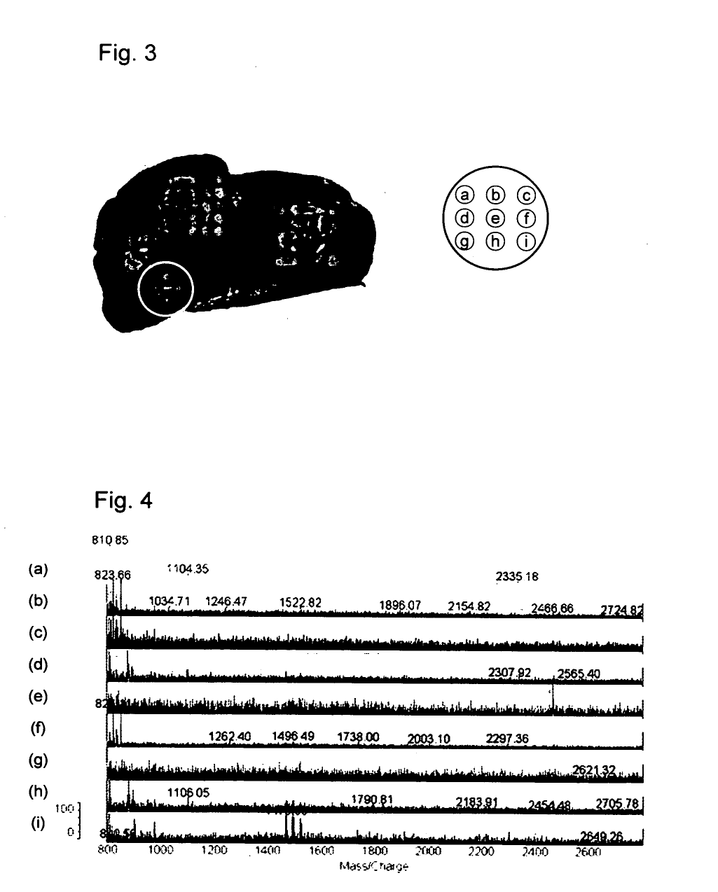

The dispensed position on the sample shows in FIG. 3. The left photograph is of the sample on which the reagent is dispensed. The part marked with a circle is enlarged to show the right illustration. As the right illustration of FIG. 3 shows, nine microscale regions a to i is set in the part marked with the circle, and each region is positioned at intervals of 500 μm. Each dispensed region was directly analyzed by MALDI-TOF mass spectrometer, AXIMA-CFR (SHIMADZU CORPORATION).

The resulting mass spectra are shown in FIG. 4(a) to (i). These letterings (a) to (i) correspond to a to i representing the nine regions shown in FIG. 3. In each figure of FIG. 4(a) to (i), horizontal axis indicates the mass-to-charge ratio of the ions (Mass/Charge), whereas vertical axis indicated the relative intensity of the ion peaks. As FIG. 4 shows, each of the close positioned microscale regions generated the independent spectrum. From the aforementioned result, the method according to the present invention can be used with effective for the analysis of microscale region in a tissue section samples.

As this invention may be embodied in several forms without departing from the spirit or essential characteristics thereof, the foregoing example is therefore only illustrative and should not be interpreted as restrictive, and all changes that fall within equivalence of claims are therefore intended to be embraced by the claims.

What is claimed is:

1. A method for dispensing a reagent onto a biological sample, wherein a reagent solution is delivered, by using inkjet technology, to a particular region on a biological sample containing cells or tissues, to make the particular region spotted with the reagent.

2. The method according to claim 1, wherein microstructures of the cells or the tissues are preserved in the same forms as are found in vivo.

3. The method according to claim 1, wherein the cells and the tissues of the biological sample are embedded in an embedding material.

4. The method according to claim 1, wherein the embedding material is selected from the group consisting of water, paraffin, celloidin, carbowax, gelatin, albumin, agarose, epoxy resin, polyester resin, and glycol methacrylate.

5. The method according to claim 1, wherein the reagent is a compound that chemically reacts with a target material present in the cells or the tissues and/or a compound that specifically binds to the target material present in the cells or the tissues.

6. The method according to claim 5, wherein following the delivery of the reagent solution for spotting to the biological sample, the reagent chemically or biologically reacts with the target material.

7. The method according to claim 5, wherein the compound that specifically binds to the target material present in the cells or the tissues comprises at least one biological compound selected from the group consisting of proteins, peptides, nucleic acids, sugars, and lipids, and/or an analogue of the biological compound that has the same or similar binding ability to the biological compound.

8. The method according to claim 5, wherein the compound that specifically binds to the target material present in the cells or the tissues comprises at least one biological compound selected from the group consisting of antibodies, antigens, and enzymes, and/or an analogue of the biological compound that has the same or similar binding ability to the biological compound.

9. The method according to claim 5, wherein the compound that specifically binds to the target material present in the cells or the tissues comprises at least one selected from the group consisting of labeled forms of the biological compound labeled with a labeling compound, complexes that the biological compound forms with the labeling compound, labeled forms of the analogue labeled with the labeling compound, and complexes that the analogue forms with the labeling compound.

10. The method according to claim 9, wherein the labeling compound is selected from the group consisting of fluorescent compounds, enzymes, heavy metal-containing compounds, and radioisotope-containing compounds.

11. The method according to claim 10, wherein the fluorescent compound is fluorescein isothiocyanate and/or tetramethylrhodamine isothiocyanate.

12. The method according to claim 10, wherein the enzyme is selected from the group consisting of horseradish peroxidase, alkaline phosphatase, acidic phosphatase, glucose oxidase, and tyrosinase.

13. The method according to claim 10, wherein the heavy metal-containing compound is ferritin and/or colloidal gold.

14. The method according to claim 1, wherein the biological sample is immobilized onto a surface of a substrate selected from the group consisting of glass substrate, resin substrate, and metal substrate, the biological sample is transferred to a membrane, or the biological sample is transferred to the membrane and then the membrane transferred is fixed to the surface of the substrate.

15. The method according to claim 14, wherein the metal substrate is a sample plate for mass spectrometry.

16. The method according to claim 14, wherein the membrane is a synthetic organic polymer or a derivative thereof selected from the group consisting of polyvinylidene difluoride, nitrocellulose, polyamide, and polyethylene.

17. A method for analyzing a biological sample containing cells or tissues, comprising the steps of:
dispensing a reagent onto a biological sample for delivering a reagent solution, by using inkjet technology, to a particular region on the biological sample containing cells or tissues, to make the particular region spotted with the reagent solution; and
subsequently analyzing a target material present in the cells or the tissues by microscopy or mass spectrometry.

18. The method according to claim .17, wherein the microscopic analysis includes detecting a color, luminescence, or fluorescence of the target material, or the presence of a radioisotope in the target material.

19. The method according to claim 17, wherein the mass spectrometry is laser desorption ionization mass spectrometry.
